(19) United States
(12) Patent Application Publication
(10) Pub. No.: US 2008/0269768 A1
(43) Pub. Date: Oct. 30, 2008

(54) Bone screw holding device

(75) Inventors: Manuel Schwager, Zurich (CH); Klaus Dorawa, Schoenkirchen (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Trauma SA, Selzach (CH)

(21) Appl. No.: 12/082,696

(22) Filed: Apr. 10, 2008

Related U.S. Application Data
(60) Provisional application No. 60/922,599, filed on Apr. 10, 2007.

(51) Int. Cl. A61B 17/58; B25B 13/00; B25B 13/48

(52) U.S. Cl. 606/104; 81/52; 81/436

(57) ABSTRACT

A screw holding device has a screw engaging part and a fixation sleeve. The screw for use with the screw holding device has a head and a shaft. The head of the bone screw has a recess and a notch. The screw engaging part has two halves with a hook formed on each half. The hook is shaped to fit in the notch. A fixation sleeve slides over the screw engaging part. When the fixation sleeve is pulled in proximal direction, the two halves of the screw engaging part are able to flex easily towards each other and thereby are insertable in the recess. When the fixation sleeve is moved towards the distal end of the screw engaging part, the two halves of the screw engaging part are prevented from flexing towards each other and thereby rigidly lock the screw and the screw engaging part together.

### CROSS-REFERENCE TO RELATED APPLICATIONS

This application claims the benefit of the filing date of U.S. Provisional Patent Application No. 60/922,599 filed Apr. 10, 2007, the disclosure of which is hereby incorporated herein by reference.

### BACKGROUND OF THE INVENTION

1. Field of Invention

The present invention relates to a device and method for inserting bone screws during a surgical procedure. More particularly, the present invention relates to an improved system of a bone screw holding device and a bone screw wherein the bone screw is held rigidly prior to and during insertion.

2. Description of the Related Art

Many types of bone screws are available for use with specific type of bone tissue or orthopedic implants. Often, a surgeon needs to install a large number of bone screws. Bone screws that are not secured to the driver can slip and become lost in the tissue. This results in unnecessary and undesirable increase in operating time. Therefore, it is desirable for the bone screws to be coupled to the driver.

Prior art screw and driver system that capture the screw prior to insertion do not provide rigid attachment of the screw to the driver and therefore, have the same shortcoming as the traditional screw and screw driver. Accordingly, there is a need for a bone screw holding device that rigidly engages the bone screw prior to and during the insertion.

### SUMMARY OF THE INVENTION

The present invention overcomes the shortcomings of the prior art by providing a screw holding device. As used herein, when referring to the screw holding device or its components, the term “proximal” means closer to the user of the screw holding device (i.e., the surgeon) and the term “distal” means more distant from the user of the screw holding device (i.e., the surgeon). The screw holding device has a screw engaging part and a fixation sleeve. The screw for use with the screw holding device has a head and a shaft with threads. The head of the bone screw has a recess with six facets. Towards the distal end (i.e., towards the leading edge of the screw) of the recess and continuous with the six facets is a notch.

The screw engaging part is tubular and has at least one slot formed towards its distal end. The slots separate the tubular part into at least two halves that can flex towards and away from each other. The distal ends of the at least two halves have six facets to match the six facets in the recess. A hook is formed on each half of the screw engaging part distal to the six facets. The hook is shaped to fit in the notch when screw engaging part is inserted in the head of the screw. A handle may be formed on the proximal end of the screw engaging part.

The fixation sleeve has a distal end, a proximal end and a tubular body connecting the distal end and the proximal end. The fixation sleeve is hollow, the hollow portion having a diameter sufficiently large to allow the screw engaging part to smoothly slide in the fixation sleeve. A disk may be formed at the proximal end of the fixation sleeve which may be used to assist in pushing or pulling the fixation sleeve with respect to the screw engaging part.

The screw engaging part and the fixation sleeve are assembled together to form the screw holding device. In the assembled state, the screw engaging part and the fixation sleeve slide relative to each other. When the fixation sleeve is moved towards the proximal direction, the two halves of the screw engaging part are able to flex easily towards each other. When the fixation sleeve is moved towards the distal end of the screw engaging part, the two halves of the screw engaging part are prevented from flexing towards each other.

In use, the fixation sleeve may slide in the proximal direction and the distal tip of the screw engaging part may be pressed against the edges of the recess, thereby inserting the distal tip of the screw engaging part into the recess. When the distal tip of the screw engaging part is pressed against the edges of the recess, the two halves of the screw engaging part are pushed towards each other allowing the distal tip of the screw engaging part the ability to enter the recess. As the tip of the distal part of the screw engaging part travels further in the distal direction into the recess, it reaches the notch and the hooks engage in the notch allowing the two halves of the screw engaging part to flex away from each other. The seating of the hooks in the notch prevents the screw holding device from disengaging from the screw accidentally. At this point, the fixation sleeve positioned distally as far as possible to prevent the two halves of the screw engaging part from flexing towards each other and thereby ensuring that the screw is securely attached to the screw holding device.

### BRIEF DESCRIPTION OF THE DRAWINGS

FIG. 1 is a cross sectional view of a screw holding device engaged with a screw.

FIG. 2 is an enlarged view of a portion of FIG. 1 showing details of engagement between the screw holding device and the screw.

FIG. 3 is a cross sectional view of the screw of FIG. 1.

FIG. 4 is an enlarged view of the head portion of the screw of FIG. 3.

FIG. 5 is an isometric view showing a screw engagement part.

FIG. 6 is an enlarged view of the distal tip of the screw engagement part of FIG. 5.

FIG. 7 is a side view of the screw engagement part of FIG. 5.

FIG. 8 is an enlarged view of the distal tip of the screw engagement part of FIG. 7.

FIG. 9 is an isometric view of a fixation sleeve.

FIG. 10 is an enlarged view of the distal tip of the fixation sleeve of FIG. 9.

FIG. 11 is an isometric view of a screw holding device engaged with a screw.

FIG. 12 is a side view of a screw.

FIG. 13 is an isometric view of a screw.

FIG. 14 is an isometric view of a screw engagement part.

FIG. 15 is an isometric view of a screw holding device.

FIG. 16 is an isometric view of a fixation sleeve.

FIG. 17 is another isometric view of a fixation sleeve.

FIG. 18 shows an isometric view of the distal portion of a screw engaging part and a bone screw.

FIG. 19 shows an isometric view of the distal portion of a screw engaging part, the distal portion of a fixation sleeve, and the proximal portion of a bone screw.

FIG. 20 shows a cross-sectional view of the distal portion of a screw engaging part and the proximal portion of a bone screw.

FIG. 21 shows a cross-sectional view of the distal portion of a screw engaging part connected to the proximal portion of a bone screw.

FIG. 22 shows an isometric view of the distal portion of a screw holding device and a bone screw.

FIG. 23 shows an isometric view of the distal portion of a screw holding device connected to a bone screw.

FIG. 24 shows a cross-sectional view of the distal portion of a screw holding device connected to a bone screw.

FIG. 25 shows another view of the distal portion of a screw holding device connected to the proximal portion of a bone screw.

### DETAILED DESCRIPTION

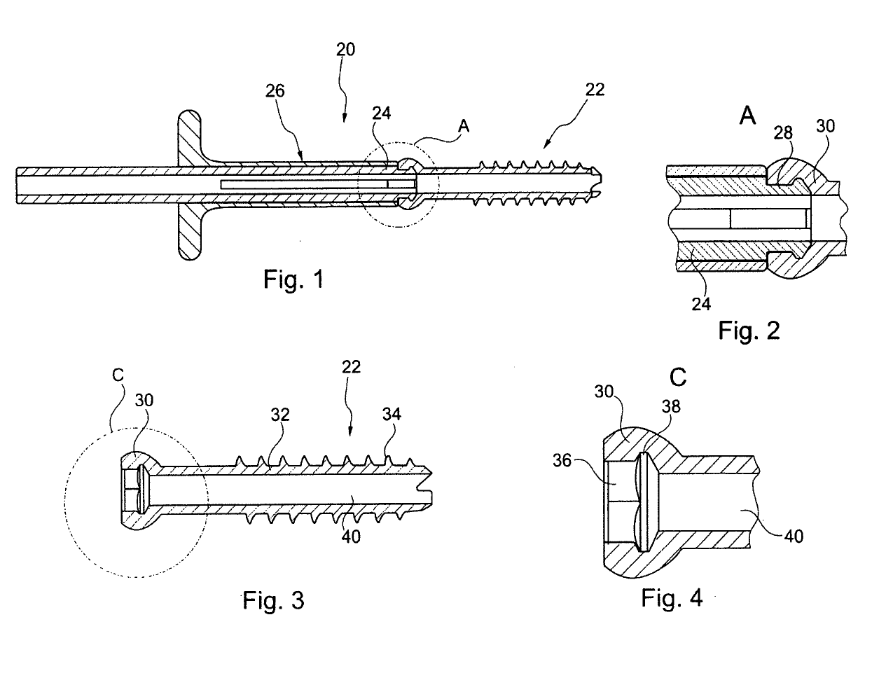

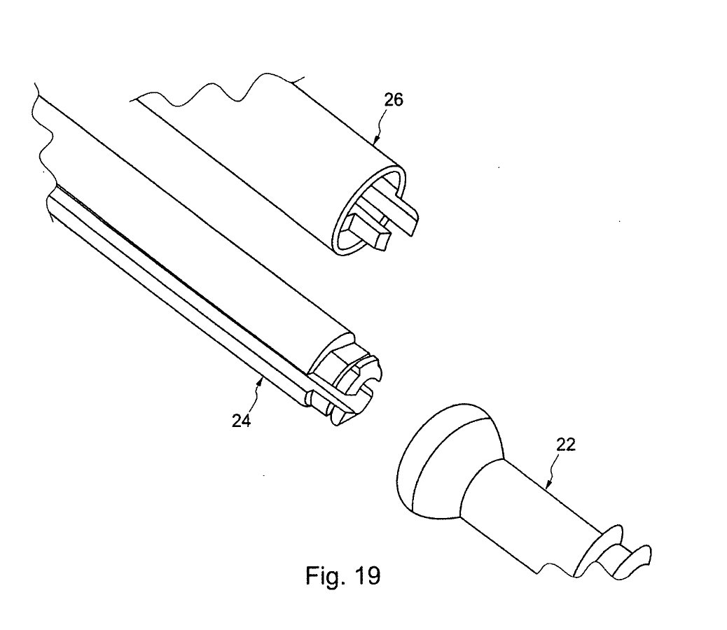

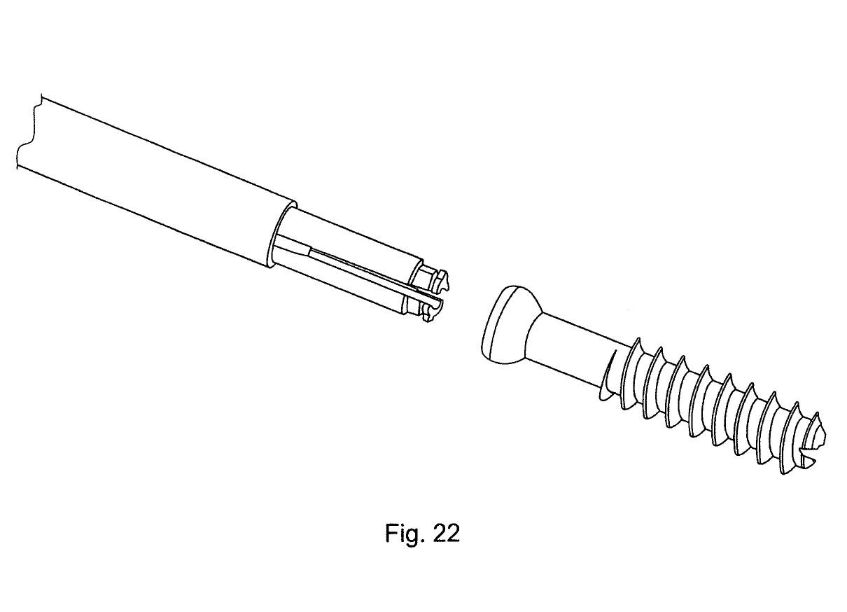

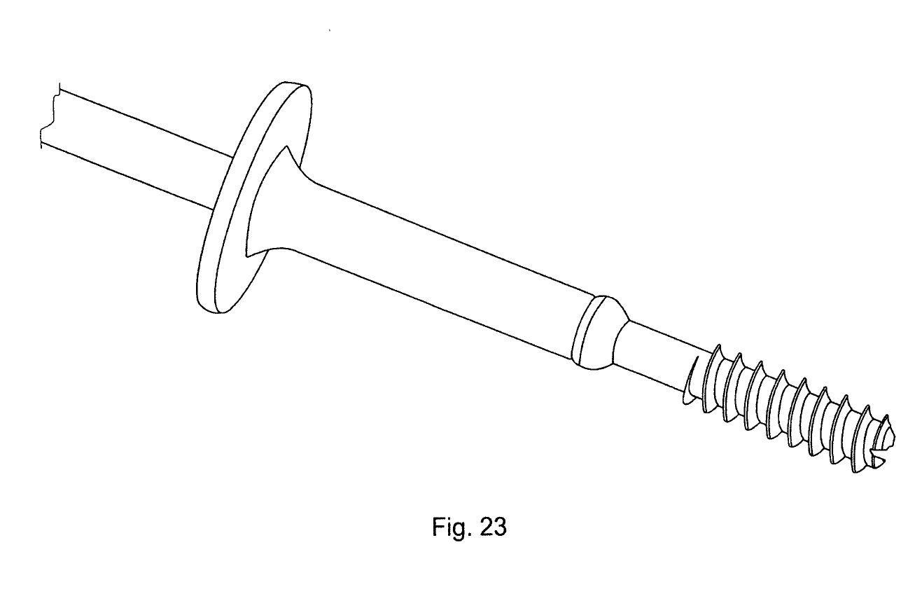

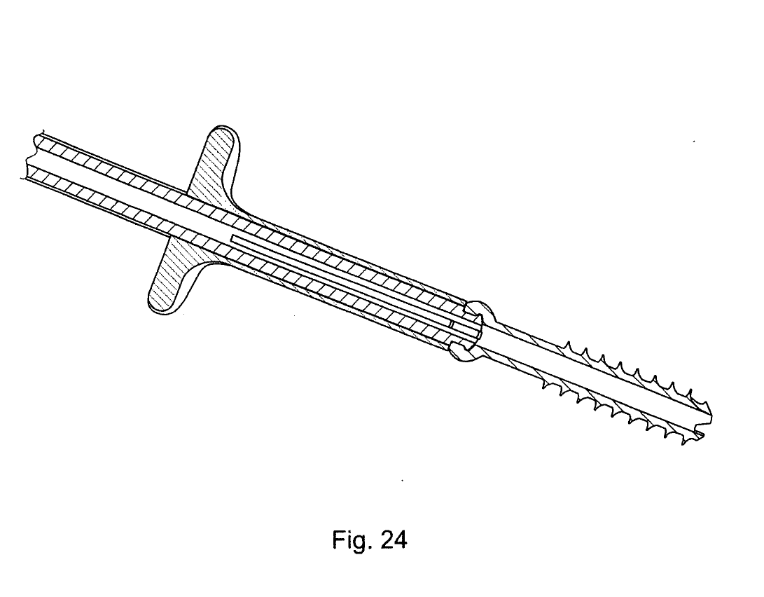

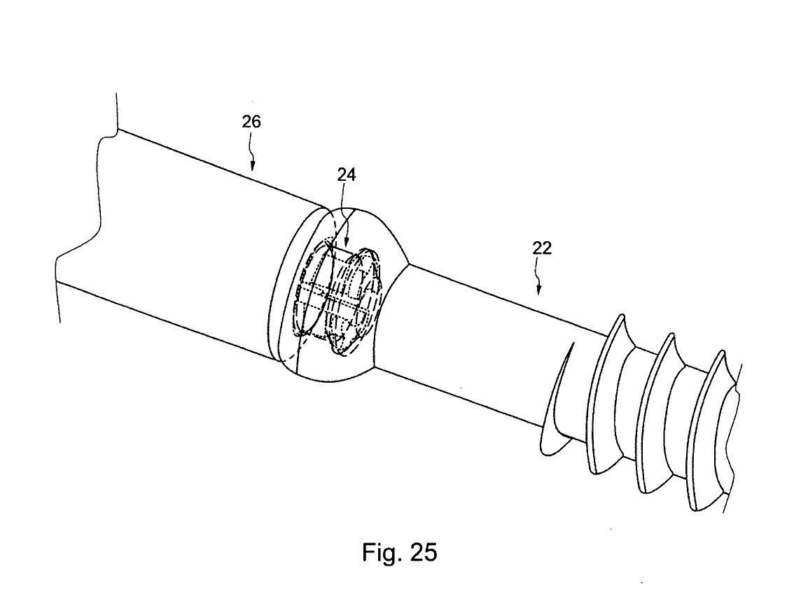

FIGS. 1 and 11 show a screw holding device 20 engaged with a screw 22. FIGS. 18-25 depict various components of screw holding device 20 in various state of assembly and in engagement with screw 22. Screw holding device 20 has a screw engaging part 24 and a fixation sleeve 26. FIG. 2 is an enlarged view of the distal portion of screw engaging part 24 and screw 22. FIG. 2 shows a tip 28 of screw engaging part 24 inserted into a head 30 of screw 22.

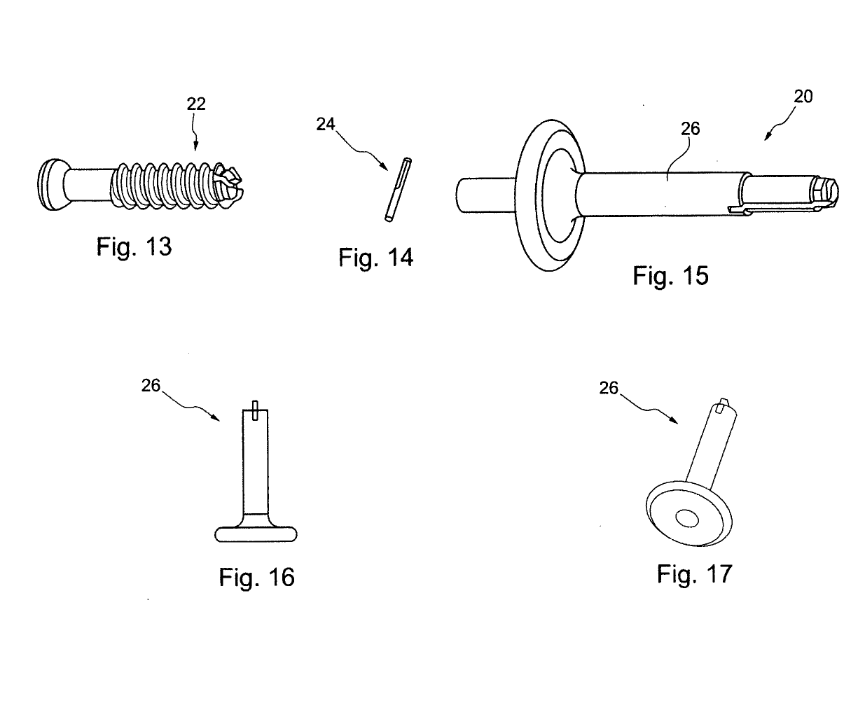

Screw 22 is depicted in a side view in FIG. 12, an isometric view in FIG. 13, and cross-sectional views in FIGS. 3 and 4. Screw 22 includes head 30 and a shaft 32 having threads 34. Threads 34 may be selected from the various types of threads known to one skilled in the art. Screw 22 may be used as a bone screw. Head 30 has a recess 36 having six facets. However, recess 36 may be of any suitable shape known to one skilled in the art, non-limiting examples of which include any type of polygon, oval, oblong or star shape. There may be more or less than six facets, and such facets may be plane, concave, or convex. Towards the distal end of recess 36 and continuous with the six facets is a notch 38 that may have a diameter slightly larger then the root diameter of the six facets. The portion of recess 36 distal to notch 38 is tapered to a smaller diameter and preferably connects with a tubular opening 40 that extends at least some distal distance along the length of screw 22.

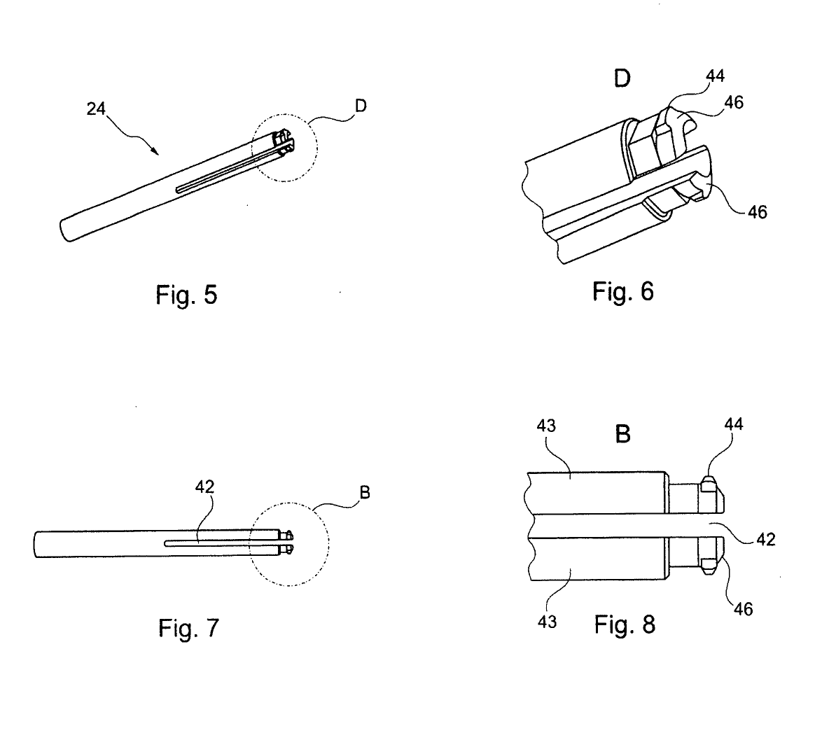

FIGS. 5, 6, 7, 8 and 14 show screw engaging part 24 which is tubular and has two slots 42 formed towards its distal end. Slots 42 separate the distal portion of screw engaging part 24 into two arms 43 that may flex towards and away from each other. Combined, the distal ends of the two arms 43 have six facets to match the six facets in the recess 36. The distal ends of the two arms 43 may be of any suitable shape known to one skilled in the art in conjunction with the configuration of recess 36. The mating surfaces of recess 36 and the distal ends of arms 43 are preferably defined by facets, but may be any such configuration that prevents rotational movement between recess 36 and the distal ends of arms 43 when such are connected. A hook 44 is formed on each arm 43 distal to the six facets. Each hook 44 is shaped to fit in notch 38 when screw engaging part 24 is inserted into head 30. Hook 44 and notch 38 can be of any suitable matable shapes, for example, the trapezoidal cross-section as seen in FIGS. 4 and 6. Alternatively, hook 44 may be provided on screw 22 and notch 38 may be provided on each arm 43. A distal face 46 of each arm 43 is tapered to form a pointed end. The proximal end of screw engaging part 24 is suitably formed to attach to a drive means, for example, a sonotrode. Alternatively, a handle may be formed on the proximal end of screw engaging part 24. Screw engaging part 24 may be made from any suitable material, an example of which is stainless steel, titanium, or a titanium alloy.

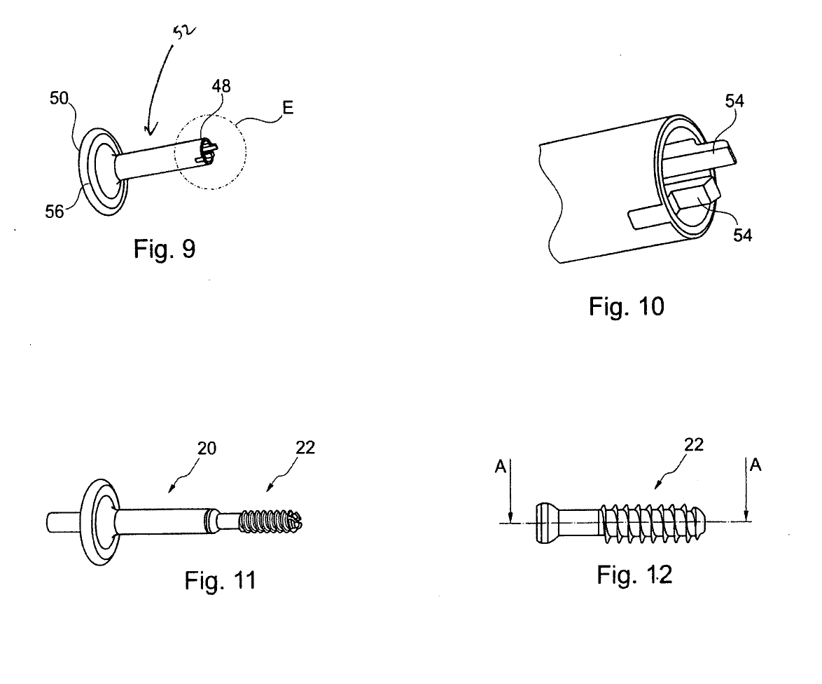

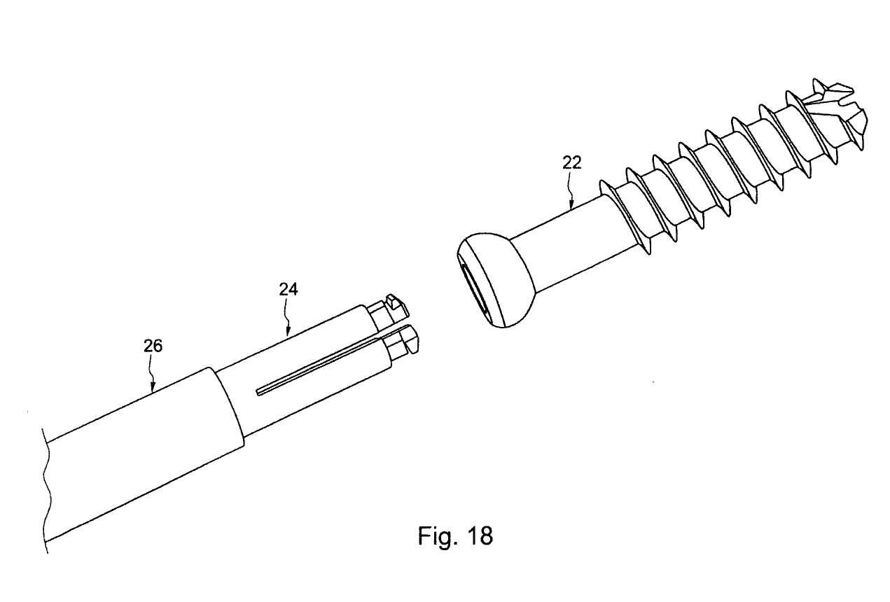

FIGS. 9, 10, 16 and 17 show fixation sleeve 26 having a distal end 48, a proximal end 50, and a tubular body 52 connecting distal end 48 and proximal end 50. Fixation sleeve 26 is hollow having an inner diameter sufficiently large to allow screw engaging part 24 to smoothly slide into fixation sleeve 26. Fixation sleeve 26 may be made from any suitable material, an example of which is stainless steel, titanium, or a titanium alloy. Two ribs 54 are formed on the inside surface of fixation sleeve 26 at distal end 48. Ribs 54 may be diametrically opposed to each other and may extend from distal end 48. A disk 56 is preferably formed at the proximal end 50 of fixation sleeve 26 and may be used to aid in pushing pulling fixation sleeve 26 with respect to screw engaging part 24. Ribs 56 are depicted having a rectangular cross section, although having such shape is not necessary. Ribs 56 may take on any suitable shape so as to be complimentary to slots 42. When fixation sleeve 26 is connected to screw engaging part 24, ribs 56 are disposed within slots 42 so as to effectively guide the translational movement of fixation sleeve 26 relative to screw engaging part 24. Moreover, the width of ribs 56 is substantially the same as the circumferential width of slots 42, such that when ribs 56 are disposed within slots 42, the circumference of that portion of screw engaging part is substantially complete and rigid.

As depicted in FIG. 15, screw engaging part 24 and fixation sleeve 26 are assembled to form screw holding device 20. When screw holding device 20 is assembled, ribs 56 are positioned in slots 42 and screw engaging part 24 and fixation sleeve 26 slide relative to each other. The movement of the screw engaging part 24 relative to fixation sleeve 26 is limited by the length of slots 42 in a proximal-distal direction. When fixation sleeve 26 is pulled in a proximal direction with respect to screw engaging part 24, ribs 56 move towards the proximal end of slots 42, leaving the distal portions of slots 42 unoccupied and thereby allowing arms 43 to flex towards each other. When fixation sleeve 26 is moved towards the distal end of screw engaging part 24, ribs 56 are positioned in the distal area of the slots 42 and prevent arms 43 from flexing towards each other.

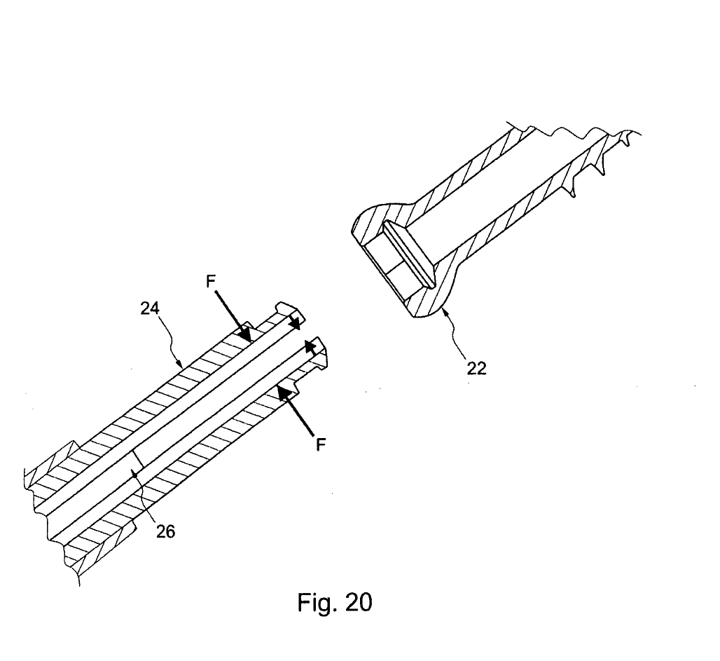

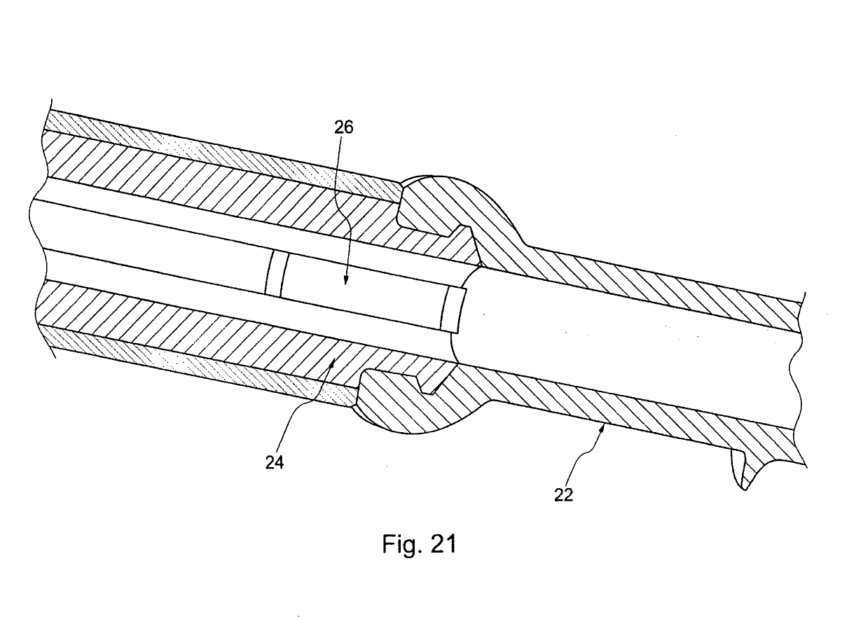

FIG. 20 illustrates a position in which fixation sleeve 26 is not connected with screw engaging part 24. Thus, it is possible to flex arms 43 radially and to hook into head 30 of screw 22. It should be noted that arms or halves 43 may bend inwardly or may bend outwardly to engage, depending on whether screw 22 includes recess 36 with notch 38 (as depicted in FIGS. 1 and 2) or whether screw 22 includes an outer recess in a direction toward the longitudinal axis (not shown) where arms 43 engage from outside. FIG. 21 illustrates the closed position in which fixation sleeve 26 is moved forward and prevents radial movement of arms 43.

In use, fixation sleeve 26 slides in the proximal direction and tip 28 of screw engaging part 24 is pressed against the edges of recess 36 thereby inserting tip 28 into recess 36. When tip 28 is pressed against the edges or facets of recess 36, arms 43 are flexed towards each other due to the taper formed on distal faces 46. With arms 43 pushed towards each other, tip 28 is able to enter recess 36. As tip 28 travels further in the distal direction in recess 36, hooks 44 engage in notch 38 allowing arms 43 to flex away from each other. The seating of hooks 44 in notch 38 aides in preventing screw holding device 20 from accidentally disengaging from screw 22. At this point fixation sleeve 26 is moved distally as far as possible with respect to screw engaging part 24 in order to prevent arms 43 from flexing towards each other, and thereby ensuring that screw 22 is securely attached to screw holding device 20.

Screw holding device 20 may be detached from screw 22 by moving fixation sleeve 26 proximally along screw engaging part 24. This movement allows arms 43 the ability to flex towards each other. When a sufficient proximal force is then applied to screw engaging part 24, hooks 44 may disengage from notch 38 thereby separating screw holding device 20 from screw 22.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An orthopedic screw holding and insertion system comprising:
a bone screw having a head, a shaft connected to the head, and threads formed on at least a portion of the shaft, the head having a recess; and
a bone screw holding device having a screw engaging part and a fixation sleeve slidably connectable to the screw engaging part, the screw engaging part including a distal end and a proximal end, the distal end having at least one slot extending towards the proximal end thereby forming a first arm and a second arm;
wherein the first arm and the second arm are moveable towards each other when the fixation sleeve is located towards the proximal end of the screw engaging part to allow insertion of the first arm and the second arm in the recess, and the first arm and the second arm are immovable with respect to each other when the fixation sleeve is located towards the distal end of the screw engaging part thereby locking the first arm and the second arm in the recess.

2. The system of claim 1 wherein the fixation sleeve includes at least one rib connectable to the at least one slot of the screw engaging part.

3. The system of claim 2 wherein the screw engaging part has two slots and the fixation sleeve has two ribs connectable to the two slots.

4. The system of claim 1 wherein the shaft defines a tubular opening.

5. The system of claim 1 wherein at least a portion of the recess comprises at least two recess facets and the distal end further comprises at least two device facets configured to engage the recess facets.

6. The system of claim 1 wherein the recess further comprises a notch and wherein the distal end further comprises at least one hook configured to engage the notch.

7. The system of claim 1 wherein the screw engaging part is attachable to a drive means.

8. The system of claim 1 wherein the proximal end of the screw engaging part includes a handle.

9. The system of claim 1 wherein the fixation sleeve comprises a disk on the proximal end.

10. An orthopedic screw holding device for holding a bone screw comprising:
a screw engaging part including a distal end and a proximal end, the distal end having at least one slot extending towards the proximal end thereby forming a first arm and a second arm; and
a fixation sleeve slidably connectable to the screw engaging part;
wherein the first arm and the second arm are moveable towards each other when the fixation sleeve is located towards the proximal end of the screw engaging part to allow insertion of the first arm and the second arm into the head of the screw, and the first arm and the second arm are immovable with respect to each other when the fixation sleeve is located towards the distal end of a screw engaging part thereby locking the first arm and the second arm into the head of the screw.

11. The system of claim 10 wherein the fixation sleeve includes at least one rib connectable to the at least one slot of the screw engaging part.

12. The system of claim 11 wherein the screw engaging part has two slots and the fixation sleeve has two ribs connectable to the two slots.

13. The system of claim 10 wherein the screw engaging part is attachable to a drive means.

14. The system of claim 10 wherein the proximal end of the screw engaging part includes a handle.

15. The system of claim 10 wherein the fixation sleeve comprises a disk on the proximal end.

16. A method of inserting a bone screw comprising:
providing a bone screw, the bone screw having a head, a shaft connected to the head, and threads formed on at least a portion of the shaft, the head having a recess;
providing a bone screw holding device, the bone screw holding device having a screw engaging part and a fixation sleeve slidably connectable to the screw engaging part, the screw engaging part including a distal end and a proximal end, the distal end having at least one slot extending towards the proximal end thereby forming a first arm and a second arm;
wherein the first arm and the second arm are moveable towards each other when the fixation sleeve is located towards the proximal end of the screw engaging part to allow insertion of the first arm and the second arm in the recess, and the first arm and the second arm are immovable with respect to each other when the fixation sleeve is located towards the distal end of the screw engaging part thereby locking the first arm and the second arm in the recess;
sliding the fixation sleeve towards the proximal end of the screw engaging part;
inserting the first and second arms into the recess such that the screw engaging part and the bone screw are fixedly connected;
sliding the fixation sleeve towards the distal end of the screw engaging part; and
turning the screw holding device to insert the bone screw in a bone.

17. The method of claim 16 further comprising:
sliding the fixation sleeve towards the proximal end of the screw engaging part; and
releasing the screw holding device from the screw.
